(19) United States
(12) Patent Application Publication
(10) Pub. No.: US 2008/0311323 A1
(43) Pub. Date: Dec. 18, 2008

(54) Cyanoacrylate Adhesive Compositions and Devices and Process for Sterilization Thereof

(75) Inventor: Carlos R. Morales, Cumming, GA (US)

Correspondence Address: ALTIMATIA, L.L.C., 16 ELM RIDGE ROAD, PENNINGTON, NJ, 08534, US

(73) Assignee: CHEMENCE, INC., Alpharetta, GA (US)

(21) Appl. No.: 11/762,018

(22) Filed: Jun. 12, 2007

(51) Int. Cl. B65D 85/00; A61L 2/04

(52) U.S. Cl. 428/35.7; 422/1

(57) ABSTRACT

Disclosed are processes for sterilization of cyanoacrylate adhesive compositions; the compositions, comprising 2-cyanoacrylate ester monomers, so produced and a method for assaying the effectiveness of the sterilization process. A typical process comprises heating the adhesive composition to from about 70° C. to about 140° C. for an effective amount of time. The effectiveness of a process can be assayed by disposing bacterial spores in the cyanoacrylate monomer, exposing the composition to a dry heat sterilization process, transferring the cyanoacrylate composition to a sterile aldose solution, transferring and exposing the sample to a nutrient medium which supports germination and growth of viable spores, incubating the samples, and determining the presence or absence of growth.

### FIELD

Embodiments of the invention relate to compositions of cyanoacrylate monomer and polymer adhesive compositions, processes for sterilizing cyanoacrylate compositions for application in the medical and veterinary fields, and a method of assaying the sterilization of cyanoacrylate compositions.

### BACKGROUND

It is known to use 2-cyanoacrylate esters as adhesives for bonding tissue in medical or surgical procedures performed upon the human or animal body. 2-cyanoacrylate esters polymerize rapidly, and often instantaneously, upon contact with tissue or body fluid. In these applications, the adhesive composition can be used to close wounds, as well as for covering and protecting surface injuries such as lacerations, abrasions, burns, sores and other open surface wounds. To be used in medical and veterinary fields, 2-cyanoacrylates must be sterilized. This is generally done in sealed containers to provide sterility, and from a practical perspective, to protect the compositions from moisture and premature polymerization. Previous sterilization methods involved either the use of ionizing radiation, including e-beam and gamma ray irradiation, dry heat at elevated temperatures (160° C.), or chemical sterilization such as with ethylene oxide.

When an adhesive composition is applied to a surface to be closed or protected, it is usually in its monomeric form, and the resultant polymerization produces the desired adhesive bond. However, at ordinary temperatures, the monomeric form of the adhesive has a low viscosity which results in the adhesive spreading into undesired areas. Therefore, it is desirable to increase the viscosity of the composition to prevent this unwanted flow. In order to achieve an increased viscosity, thickening agents can be added to the monomeric composition.

The previous methods of sterilization are undesirable in that the high temperatures required for the previous dry heat sterilization processes or irradiation could cause premature polymerization of the monomers. In addition, many polymers that could be used as thickeners underwent degradation resulting in loss of viscosity when treated with ionizing radiation or to dry heat conditions of 160° C. This significantly limits the formulators ability to formulate adhesive compositions which have the desirable stability and flow characteristics, and which can be sterilized.

### SUMMARY

Embodiments of the present invention are directed to a method of sterilizing 2-cyanoacrylate compositions, including heating the composition in a device at a temperature of from about 70° C. to about 140° C. for an effective amount of time. In another aspect, embodiments of the invention include sterilized 2-cyanoacrylate ester compositions for use in medical applications or surgery, the compositions being disposed in sealed aluminum, tin, stainless steel, glass, or plastic containers and being sterilized at a temperature of between about 70° C. and about 140° C. In yet another aspect, embodiments of the invention are directed to a method for assaying the sterilization of cyanoacrylate compositions.

### DETAILED DESCRIPTION

Embodiments of the present invention provide a novel method of sterilizing 2-cyanoacrylate ester compositions using a dry heat means, and the resulting novel compositions. The combination of monomeric 2-cyanoacrylate, heat and time have a lethal effect on microbials, rendering sterilized compositions when the appropriate sterilization condition is achieved and when the method is applied to 2-cyanoacrylates in sealed containers.

As used herein, the following terms have the following meanings:

The term “cyanoacrylate adhesive composition” or “cyanoacrylate adhesive compositions” refers to polymerizable formulations comprising polymerizable cyanoacrylate ester monomers. The term aldose is intended to refer to both common disaccharides and monosaccharides.

In a method of the invention, 2-cyanoacrylate adhesive compositions are sterilized through an unexpected and heretofore unknown combination of heat and time, sterilizing at temperatures significantly lower than previously thought to be effective. Previous dry heat sterilization methods have required temperatures of at least 160° C. to 180° C. Heating times at these temperatures were from 2 hours at 160° C. to 30 minutes at 180° C. Under the present invention, the 2-cyanoacrylate adhesive compositions can be sterilized at temperatures from about 70° C. to about 140° C. As would be expected, the time required to effect sterilization will vary depending on the temperature selected to accomplish the sterilization. At 140° C., sterilization requires approximately 30 minutes. At 70° C., sterilization requires about 600 minutes. Required heating times for intermediate temperatures are reported in Tables 2 and 3. Ultimately sterilization times for any composition can be readily determined by one skilled in the art by standard test methods without undue experimentation. Typical sterilization times are listed in Table 1.

TABLE #1
sterilization heating times

| | |
|---|---|
| 70° C. | 600 minutes |
| 80° C. | 480 minutes |
| 90° C. | 300 minutes |
| 100° C. | 120 minutes |
| 110° C. | 90 minutes |
| 120° C. | 60 minutes |
| 130° C. | 60 minutes |
| 140° C. | 30 minutes |

According to an assay method of the invention, sterilization of cyanoacrylate compositions can be assayed for the effectiveness of a given temperature and sterilization time. Samples containing formulated n-butyl cyanoacrylate and 2-octyl cyanoacrylate in sealed borosilicate glass, aluminum tubes, and high density polyethylene (HDPE) containers were inoculated with Bacillus subtillis lyophilized spores at a concentration of 1×10⁺⁶ per ml of formulation. In other embodiments, spores can be introduced into the cyanoacrylate adhesive compositions prior to sterilization using commercially available biological indicators or spore test strips. Among the commercially available biological indicators which may be used are: bacterial spores on a stainless steel disc, bacterial spores on a steel wire, bacterial spores on steel coupons, bacterial spores on borosilicate paper and bacterial spores on woven cotton threads. Among the species of spores which may be chosen for use in the commercially available biological indicators are Bacillus subtillis and Geobacillus Stearothermophillus. Commercially available biological indicators may be obtained from any commercial supplier, such as Raven Labs. Some inoculated glass vial and tube samples were kept at room temperature without sterilization as positive controls, while the rest of the samples were sterilized at temperatures ranging from 70 to 140° C. with different time exposures. Samples were sent to a microbiology laboratory for determination of the presence or absence of growth after the sterilization procedure was completed to assay the effectiveness of the process conditions.

In accordance with embodiments of the present invention it is preferred to utilize microorganisms which may be killed by the sterilization process but which show significant resistance to this process. The term microorganism refers to bacteria, fungi, yeast, protozoa algae, viruses and protozoa. Bacterial spores are very resistant to heat and chemicals; more so than vegetative bacterial cells, therefore the spores are often used to monitor sterilization procedures. A preferred organism for monitoring dry heat sterilization is Bacillus subtillis.

The spores represent a resting stage in the life cycle of the Bacillus genus. The resting spore contains a large number of active enzymes which allow the transformation from dormant cell to vegetative cell. The germination process, or the return to the vegetative state, has been described as a time-ordered sequence involving activation, triggering, initiation, and outgrowth. Activation is reversible and involves an increase in the rate and extent of germination. Triggering is irreversible and is the result of spore contact with the germinant. Initiation involves the loss of heat resistance, release of dipicolinic acid and calcium, loss of refractility and absorbance. Outgrowth results in formation of the vegetative cell.

In accordance with embodiments of the present invention a cyanoacrylate composition test sample comprising at least one sterility test strip, or lyophilized spores is utilized. While reference is made to “spores” as a test microorganism it should be understood that microorganisms other than spore formers may be used in conjunction with the present invention. The spore strips utilized with the present invention are preferably constructed of a materials which is inert to the microorganisms and inert to cyanoacrylate monomer. A variety of commercial spore strips are readily available and can be utilized with the present invention. The spore strips can contain more than one type of microorganism.

To assay the sterilized samples and controls, the compositions including the biological indicators are transferred into containers filled with an aqueous aldose solution, shaken, and transferred into a quantity of nutrient medium in an aseptic container. Transferring the samples to an aldose solution serves to emulsify the cyanoacrylate monomer without causing it to polymerize as it would upon exposure to water alone. Aldoses which act to emulsify the cyanoacrylate include without limitation, dextrose, lactose, arabinose, mannose, galactose, rhamnose, fructose, sucrose, and glucose. In one embodiment of the invention, the aldose is dextrose. The concentration of the aldose solution may be from about 2% to about 50% on a weight/weight basis. A preferred range for the concentration of the aldose solution is from about 3% to about 15%. A more preferred aldose concentration is from about 5% to about 10% weight/weight. The nutrient medium supports the germination of spores and growth of any viable microorganisms. The nutrient medium contains a protein substrate for the proteases liberated during spore germination and during subsequent microbial growth. The nutrient medium preferably comprises an aqueous solution or suspension of nutrient components (including the protein substrate) needed in order to promote the growth of viable microorganisms that may exist after the sterilization process. One example of a suitable culture medium is a protein containing microbiological broth such as tryptic soy broth (TSB) and/or TSB with specific protein additives, such as, for example casein. Formulations for culture media are well-known to those in the art.

The mixture of microorganisms, cyanoacrylate, aldose, and nutrient medium are sealed within a containing means. The samples are incubated for a predetermined period of time at from about 28° C. to about 37° C. Any microorganisms not killed during the sterilization process begin to germinate and grow during the incubation period. In a preferred embodiment the samples are incubated for at least about seven days. Thereafter the samples are examined to detect the presence of microorganism growth by different methods, such as visual examination of the samples followed by microscope Gram stain examination, addition of an enzymatic indicator such as tetrazolium salts followed by UV spectrophotometric analysis, or direct UV spectrophotometric analysis of incubated samples. In one embodiment, after visual examination a gram stain smear is prepared to look for gram positive rods which would confirm microorganism growth. In another embodiment, microorganism growth can be determined by the addition of enzymatic biological indicator such as tetrazolium salts, wherein microorganism activity is determined by development of color which may be measured quantitatively with a ultraviolet spectrophotometer at 257 nm. In yet another embodiment, a sample without enzymatic indicator, is analyzed under a spectrophotometer at a wavelength of 480 nm to determine microorganism growth.

A method of the invention can be applied in principle to any 2-cyanoacrylate ester monomer. The 2-cyanoacrylate is preferably an aliphatic cyanoacrylate ester and preferably an alkyl, cycloalkyl, alkenyl, alkoxyalkyl, fluroroalkyl, fluorocyclic alkyl or fluoroalkoxy 2-cyanoacrylate ester. The alkyl group may contain from 2 to 12 carbon atoms, and is preferably a C₂ to C₈ alkyl ester, and is most preferably a C₄ to C₈ alkyl ester. Suitable 2-cyanoacrylate esters include without limitation, the ethyl, n-propyl, iso-propyl, n-butyl, pentyl, hexyl, cyclohexyl, heptyl, n-octyl, 2-ethylhexyl, 2-methoxyethyl and 2-ethoxyethyl esters. Any of these 2-cyanoacrylate monomers may be used alone, or they may be used in mixtures.

The 2-cyanoacrylate monomers of the invention can be prepared by any of the methods known in the art. U.S. Pat. Nos. 2,721,858, 3,254,111 and 4,364,876 each of which is hereby incorporated herein in its entirety by reference, disclose methods for preparing 2-cyanoacrylates. For example, cyanoacrylates for the instant invention were prepared by reacting cyanoacetate with formaldehyde in the presence of heat and a basic condensation catalyst to give a low molecular weight polymer. A depolymerization step followed under heat and vacuum in the presence of acidic and anionic inhibitors, yielding a crude monomer that could be distilled under vacuum and in the presence of radical and acidic inhibitors. The distilled 2-cyanoacrylate monomers are then formulated with radical and acidic inhibitors depending upon their application and to provide the necessary stability.

The 2-cyanoacrylate compositions of the invention may in some embodiments contain a thickening agent to increase the viscosity of the composition. This thickening agent may be a polymer. The thickening agent may be selected from the group consisting of without limitation, poly alkyl-2-cyanoacrylates, poly cycloalkyl-2-cyanoacrylates, poly fluoroalkyl-2-cyanoacrylates, poly fluorocycloalkyl-2-cyanoacrylates, poly alkoxyalkyl-2-cyanoacrylates, poly alkoxycycloalkyl-2-cyanoacrylates, poly fluoroalkoxyalkyl-2-cyanoacrylates, polyalkoxycyclofluoroalkyl-2-cyanoacrylates, poly vinylacetate, poly lactic acid and poly gylcolic acid. In order to obtain optimum solubility of the polymer in the monomer, the polymer is often chosen to be a polymer of the monomer or one of the monomers which comprise the 2-cyanoacrylate composition. Preferably, the polymer is soluble in the monomer composition at ambient temperature. Preferred polymers include polymers of octyl 2-cyanoacrylate, vinyl acetate lactic acid, or glycolic acid. The preferred weight average molecular weight of the polymers is from about 300,000 to about 2,000,000. More preferably, the polymer molecular weight is from about 500,000 to about 1,600,000.

Cyanoacrylate polymers of the invention can be prepared by slow addition of the monomer to a mixer containing 0.1% Bicarbonate deionized water. Water is then decanted away, and the polymer is rinsed several times with deionized water and decanted again. Following steps include neutralizing the polymer with 0.1N HCl, rinsing with deionized water, drying in a vacuum heated oven at temperature of less than 80° C. and grinding the polymer to fine particles.

The amount of thickening agent that is added to the monomer composition is dependent upon the molecular weight of the polymer, and the desired viscosity for the adhesive composition. The thickening agent typically is added at from about 1% to about 25% by weight of the composition. Preferably it is added at from about 1% to about 10%. More preferably it is added at from about 1% to about 5%. A typical viscosity of the composition is from about 25 to about 3000 centipoise, as measured by a Brookfield viscometer at 25° C. Preferably, the viscosity is between from about 50 to 600 centipoise at 25° C. The specific amount of a given thickening agent to be added can be determined by one of ordinary skill in the art without undue experimentation.

The 2-cyanoacrylate compositions may contain one or more acidic inhibitors in the range from 1 to 1,000 ppm. Such acidic inhibitors include without limitation: sulfur dioxide, nitrogen oxide, boron-oxide, phosphoric acid, ortho, meta, or para-phosphoric acid, acetic acid, benzoic acid, cyanoacetic acid, tri-fluoroacetic acid, tribromoacetic acid, trichloroacetic acid, boron trifluoride, hydrogen fluoride, perchloric acid, hydrochloric acid, hydrobromic acid, sulfonic acid, fluorosulfonic acid, chlorosulfonic acid, sulfuric acid, and toluenesulfonic acid.

The 2-cyanoacrylate compositions may contain one or more free radical polymerization inhibitors in the range from 0 to 10,000 ppm. Examples such radical inhibitors include without limitation, catechol; hydroquinone; hydroquinone monomethyl ether and hindered phenols such as butylated hydroxyanisol; butylated hydroxytoluene (2,6-di-tert-butyl butylphenol and 4-methoxyphenol); 4-ethoxyphenyl; 3 methoxyphenol; 2-tert-butyl-4methoxyphenol; 2,2methylene-bis-(4-methyl-6-tert-butylphenol).

The 2-cyanoacrylate compositions may contain single or mixtures of plasticizers such as tributyl acetyl citrate; dimethyl sebacate; diethyl sebacate; try-ethyl phosphate; tri-(2-ethylhexyl)phosphate; tri-cresyl phosphate; glyceryl triacetate; glyceryl tributyrate; dioctyl adipate; isopropyl myristate; butyl stearate; trioctyl trimellitate and dioctyl glutarate. The plasticizers may be added to the compositions in proportions of less than 50% w/w of the formulation.

The 2-cyanoacrylate compositions may contain small amounts of dyes like the derivatives of anthracene and other complex structures. Some of these dyes include without limitation, 1-hydroxy-4-[4-methylphenylamino]-9,10 anthracenedione (D&C violet No. 2); disodium salt of 6-hydroxy-5-[(4-sulfophenyl)axo]-2-naphthalene-sulfonic acid (FD&C Yellow No. 6,); 9-(o-carboxyphenyl)-6-hydroxy-2,4,5,7-tetraiodo-3H-xanthen-3-one,disodium salt, monohydrate (FD&C Red No. 3); 2-(1,3dihydro-3-oxo-5-sulfo-2H-indole-2-ylidine)-2,3-dihydro-3-oxo-1H-indole-5-sulfonic acid disodium salt (FD&C Blue No. 2); and [phthalocyaninato (2)] copper. add in proportions of less than 50000 ppm.

The sterilized cyanoacrylate adhesive compositions of the invention may be packaged in a container made of any suitable material. Suitable materials must be heat stable and resistant up to the sterilization temperature, must provide an adequate barrier to atmospheric moisture and be compatible with the cyanoacrylate monomer or monomers. Materials meeting these requirements include metals, glass, and plastic. Suitable metals can include without limitation aluminum, tin, stainless steel, and plastics including high density polyethylene (HDPE), low density polyethylene (LDPE), polypropylenes, phenolic resins. Especially useful plastics include surface-fluorinated plastics such as surface-fluorinated HDPE, surface-fluorinated LDPE, surface-fluorinated polypropylene, and other surface-fluorinated plastics. Such plastics may be produced by the process of the Fluoro-Seal corporation (Fluoro-Seal, 16360 Park Ten Place, Suite 325, Houston, Tex. 770084-5046, www.fluoroseal.com) Metals can have different forms like pouches and tubes. Glass can be used as vials, breakable tubes or any other shape, and contained inside tubes made out of the same material, or combinations or materials including plastics. Particularly preferred materials are aluminum, type I borosilicate glass, and high density polyethylene (HDPE). Preferred aluminum tubes comprise a nozzle which is hermetically sealed by a pierceable membrane of aluminum and are filled at their end remote from the nozzle prior to closure of the open end by tight crimping. The glass vials used in this invention, are made out of borosilicate type I glass and sealed with a threaded phenolic cap with a silicone/teflon septa or sealed with an aluminum crimp cap and silicon/teflon septa. In the result, therefore, embodiments of the invention may reside in a substantially hermetically sealed aluminum container, e.g., an aluminum tube, containing a sterile 2-cyanoacrylate composition or type I glass vials hermetically sealed with a phenolic threaded and silicone/teflon septa or in plastic containers such as containers made from HDPE, surface-fluorinated HDPE, LDPE, surface-fluorinated LDPE, polypropylene, surface-fluorinated polypropylene, phenolic resins and the like.

### EXAMPLES

### Example I

Sample testing: (Sterility test method for all samples)

The method was tested by first performing the USP bacteriostasis and fungistasis test on glass vials and aluminum tubes. The sterility test was performed by obtaining spores of Bacillus subtillis var niger suspended in irrigation water at a concentration of 2.3×10⁺⁸ ml. Aliquots of 0.48 ml of these spores were placed in glass serum bottles, lyophilized and then reconstituted with 50 ml of n-butyl or 2-octylcyanoacrylate compositions to obtain a volume of 50 ml of inoculated spore solution with a concentration of 2×10⁺⁶ ml. These cyanoacrylate spore solutions were used to fill the tubes and vials for the sterilization trials at different temperatures and time and for the non sterilized (standard biological indicators) control vials and tubes. Each tube and vial was filled with a volume of 0.5 to 0.6 ml of a cyanoacrylate composition that rendered a spore concentration of 2×10⁺⁶ ml. Non sterilized BI's and sterilized spore inoculated samples, were transferred to a 5% Dextrose USP solution, shaken and transferred to soy casein digested broth (SCDB) and incubated at 35-37° C. for at least seven days. A vial of lyophilized spores with no cyanoacrylate was tested for population verification. The vial was transferred to sterile purified water and vortexed for 10 minutes. Serial dilutions of 10⁺⁴, 10⁺⁵, 10⁺⁶ were plated in duplicate using soy bean casein digest broth (SCDB) and incubated for 48 hour at 35-37° C. The 10⁺⁶ dilution yielded duplicate plates in the countable range. The final calculations showed there were 6.1×10⁺⁶ CFU/ml, or 3.1×10⁺⁷ CFU/vial.

Polymer preparation: (polymer method for samples containing polymer)

2-OCA polymer was made by adding drop by drop 30 grams of 2-OCA monomer to a blender containing 1000 ml of 0.1% Sodium bicarbonate deionized water while swirling. Bicarbonate water with the polymer was vacuum filtered on a Kitasato with a Fisherbrand #Q5 quantitative filter paper, rinsed five times with 500 ml aliquots of deionized water, decanted and polymer neutralized with 500 ml of 0.1 N Hydrochloric acid. The neutralized polymer was rinsed with three aliquots of 500 ml, decanted, dried in a vacuum oven at 80° C. and finely ground with a mixer after drying.

Sample composition preparation:

The sample of 2-OCA containing polymer was made by mixing 2-octyl cyanoacrylate (stabilized with 100 ppm of SO₂, 1000 ppm of butylated hydroxyanisole) with 3.5% of 2-OCA polymer. The polymer was dissolved in the formulated 2-OCA by heating and mixing in a round glass flask equipped with a paddle shaft and mixer at a temperature no higher than 80° C. and obtaining a viscosity of 567 Cp (measured with Brookfield DV-II at 25° C.). Then, the composition was inoculated with lyophilized Bacillus subtillis spores enough to produce a minimum concentration of 1×10⁺⁶ which were deposited in aluminum tubes and glass type I glass threaded vials. Tubes were sealed by crimping with a Kentex automatic tubes filler and sealer. The glass vials were filled with an Eppendorf automatic pipette and sealed with threaded phenol caps and silicone/teflon septa.

Some inoculated glass and tube samples were not sterilized and were used as positive standard biological indicators to indicate livable spores. The rest of the inoculated and sealed tubes and vials were exposed to the experimental temperatures and time stipulated in the sterilization testing protocol conditions.

Tables 2 and 3 show example results.

TABLE 2
2-OCA sterilization example packed in glass vials with pre-sterilization viscosity of 567 cP

| | Sterilization | Type of | Incubation | Number | Number of | Number | |
|---|---|---|---|---|---|---|---|
| Sterilization | time | media | temperature | samples | days | of | Viscosity @ |
| ° C. | minutes | 400 ml | ° C. | tested | incubated | positives | 25° C. sterile |
| 90 | 240 | SCDB | 30-35 | 3 | 7 | 1 | 566 |
| 100 | 120 | SCDB | 30-35 | 3 | 7 | 0 | 569 |
| 100 | 180 | SCDB | 30-35 | 3 | 7 | 0 | 562 |
| 110 | 60 | SCDB | 30-35 | 3 | 7 | 0 | 526 |
| 110 | 120 | SCDB | 30-35 | 3 | 7 | 0 | 452 |
| 120 | 60 | SCDB | 30-35 | 3 | 7 | 0 | 418 |
| 120 | 90 | SCDB | 30-35 | 3 | 7 | 0 | N/A |
| 130 | 60 | SCDB | 30-35 | 3 | 7 | 0 | 343 |
| 130 | 120 | SCDB | 30-35 | 3 | 7 | 0 | N/A |
| 140 | 30 | SCDB | 30-35 | 3 | 7 | 0 | 110 |
| 140 | 45 | SCDB | 30-35 | 3 | 7 | 0 | N/A |

Table #2 above shows minimum sterilization temperatures, incubation temperature, incubation time and the results obtained for samples of Bacillus subtillis spores inoculated 2-OCA containing 3.5% 2-OCA polymer (567 cP), 100 ppm SO₂ and 1000 ppm BHA.

TABLE 3
2-OCA sterilization example packed in aluminum tubes with pre-sterilization
viscosity of 567 cP

| | Sterilization | Type of | Incubation | Number | Number of | Number | Viscosity |
|---|---|---|---|---|---|---|---|
| Sterilization | time | media | temperature | samples | days | of | @ 25° C. |
| ° C. | minutes | 400 ml | ° C. | tested | incubated | positives | sterile |
| 90 | 240 | SCDB | 30-35 | 3 | 7 | 2 | 565 |
| 100 | 120 | SCDB | 30-35 | 3 | 7 | 0 | 566 |
| 100 | 180 | SCDB | 30-35 | 3 | 7 | 0 | 570 |
| 110 | 60 | SCDB | 30-35 | 3 | 7 | 0 | 526 |
| 110 | 120 | SCDB | 30-35 | 3 | 7 | 0 | 435 |
| 120 | 60 | SCDB | 30-35 | 3 | 7 | 0 | 405 |
| 120 | 90 | SCDB | 30-35 | 3 | 7 | 0 | N/A |
| 130 | 60 | SCDB | 30-35 | 3 | 7 | 0 | 351 |
| 130 | 120 | SCDB | 30-35 | 3 | 7 | 0 | N/A |
| 140 | 30 | SCDB | 30-35 | 3 | 7 | 0 | 102 |
| 140 | 45 | SCDB | 30-35 | 3 | 7 | 0 | N/A |

Table #3 above shows minimum sterilization temperatures, incubation temperature, incubation time and the results obtained for samples of Bacillus subtillis spores inoculated 2-OCA containing 3.5% 2-OCA polymer (567 cP), 100 ppm SO₂ and 1000 ppm BHA.

### Example II

Sample composition preparation:

### Sample IIA:

A sample of n-butyl cyanoacrylate (n-BCA) with a viscosity of 2.8 cP (measured with Brookfield DV-II at 25° C.) containing 100 ppm of SO₂ and 1000 ppm of Butylated Hydroxyanisole (BHA) was prepared for this example. Then, the composition was inoculated with Biological Indicator standards (BI) such as borosilicate spore discs, cotton threads and spore wires with a spores concentration of 1×10⁶ Geobacillus stearothermophilus. The spore inoculated composition was deposited in type I glass threaded vials with an Eppendorf automatic pipette and sealed with threaded phenol caps with silicone/teflon septa. Some inoculated vials were not sterilized and were used as positive standard biological indicators to indicate viable spores. The rest of the inoculated sealed vials were exposed to the experimental temperatures and time stipulated in the sterilization testing protocol conditions.

Table 4 shows example results.

TABLE 4
n-BCA monomer sterilization example in glass vials with pre-sterilization
viscosity of 2.8 cP

| | Sterilization | Type of | Incubation | Number | Number of | Number | Viscosity |
|---|---|---|---|---|---|---|---|
| Sterilization | time | media | temperature | samples | days | of | @ 25° C. |
| 100° C. | minutes | 400 ml | ° C. | tested | incubated | positives | sterile |
| Borosilicate | 240 | SCDB | 55-60 | 3 | 7 | 0 | 2.9 |
| disc | | | | | | | |
| Cotton | 240 | SCDB | 55-60 | 3 | 7 | 0 | 2.8 |
| threads | | | | | | | |
| SS wires | 240 | SCDB | 55-60 | 3 | 7 | 0 | 2.8 |
| Positive | NO | SCDB | 55-60 | 3 | 2 | 3 | 2.8 |
| control | | | | | | | |
| borosilicate | | | | | | | |
| disc | | | | | | | |
| Positive | NO | SCDB | 55-60 | 3 | 2 | 3 | 2.9 |
| Control | | | | | | | |
| cotton | | | | | | | |
| threads | | | | | | | |
| SS wires | NO | SCDB | 55-60 | 3 | 2 | 3 | 2.8 |

Table 4 above shows sterilization temperatures, incubation temperature, incubation time and the results obtained for samples of Geobacillus stearothermophilus spores inoculated n-BCA containing, 100 ppm SO₂ and 1000 ppm BHA.

### Sample II B:

A sample of n-butyl cyanoacrylate (n-BCA) with a viscosity of 2.8 cP (measured with Brookfield DV-II at 25° C.) containing 100 ppm of SO₂ and 1000 ppm of butylated hydroxyanisole (BHA) was prepared for this example. Then, the composition was inoculated with Biological Indicator standards (BI) cotton threads with a spores concentration of 1×10⁶ Bacillus subtillis. The spore inoculated composition was deposited in type I glass threaded vials with an Eppendorf automatic pipette and sealed with threaded phenolic caps with silicone/teflon septa. Some inoculated vials were not sterilized and were used as positive standard biological indicators to indicate viable spores. The rest of the inoculated sealed vials were exposed to the experimental temperatures and time stipulated in the sterilization testing protocol conditions.

Tables 5 shows example results.

TABLE #5
n-BCA monomer sterilization example in glass vials with pre-sterilization
viscosity of 2.8 cP

| | Sterilization | Type of | Incubation | Number | Number of | Number | Viscosity |
|---|---|---|---|---|---|---|---|
| Sterilization | time | media | temperature | samples | days | of | @ 25° C. |
| 100° C. | minutes | 400 ml | ° C. | tested | incubated | positives | sterile |
| Cotton | 240 | SCDB | 55-60 | 3 | 7 | 1 | 2.8 |
| threads | | | | | | | |
| Positive | NO | SCDB | 55-60 | 3 | 2 | 3 | 2.8 |
| Control | | | | | | | |
| cotton | | | | | | | |
| threads | | | | | | | |

Table #5 above shows sterilization temperatures, incubation temperature, incubation time and the results obtained for samples of Bacillus subtillis spores inoculated n-BCA containing, 100 ppm SO₂ and 1000 ppm BHA.

### Example III

Samples of polymer modified 2-octyl (2-OCA) and n-butyl (n-BCA) cyanoacrylates stabilized with 100 ppm of SO₂ and 1000 ppm of BHA were filled and sealed in plastic HDPE pipettes with cotton thread biological indicators (BI's) added with a spore population of 1×10⁶. All samples were filled using an Eppendorf automatic pipette. Two samples were not sterilized and were used as positive controls.

TABLE 6
Samples contain a thick formulated mixture of 2-octyl and butyl cyanoacrylate
monomers with Bacillus subtillis biological cotton thread indicators packed in
plastic HDPE pipettes.

| | | | | | Number | | |
|---|---|---|---|---|---|---|---|
| | | | | | of | | |
| | Sterilization | Type of | Incubation | Number | positive | Number | Number |
| Sterilization | time | media | temperature | samples | controls | of days | of |
| ° C. | minutes | 400 ml | ° C. | tested | tested | incubated | positives |
| 110 | 180 | SCDB | 30-35 | 15 | N/A | 7 | 0 |
| | | | | Lot# | | | |
| | | | | 06L1201 | | | |
| 110 | 180 | SCDB | 30-35 | 15 | N/A | 7 | 0 |
| | | | | Lot# | | | |
| | | | | 06L0601 | | | |
| No | 0 | SCDB | 30-35 | N/A | 2 | 7 | 2 |

Table 6 above shows samples with a sterilization temperature of 110° C., incubation temperature, incubation time and the results obtained for samples and controls of formulated thick 2-OCA/n-BCA, 100 ppm SO₂ and 1000 ppm BHA.

### Prophetic Examples

### Prophetic Example I

At least five samples of polymer modified 2-octyl (2-OCA) and n-butyl (n-BCA) cyanoacrylates stabilized with 100 ppm of SO₂ and 1000 ppm of BHA will be filled and sealed in plastic LDPE containers with cotton thread biological indicators (BI's) added with a spore population of 1×10⁶. All samples will be filled using an Eppendorf automatic pipette. Two samples will not be sterilized and will be used as positive controls. The samples will be subjected to a variety of sterilization times and temperatures such as those listed in the tables above. Samples will then be analyzed for the presence of active bacteria, or spores by methods such as described above. Conditions suitable for preparation of LDPE containers filled with sterile cyanoacrylates will be identified.

### Prophetic Example II

At least five samples of polymer modified 2-octyl (2-OCA) and n-butyl (n-BCA) cyanoacrylates stabilized with 100 ppm of SO₂ and 1000 ppm of BHA will be filled and sealed in plastic polypropylene containers with cotton thread biological indicators (BI's) added with a spore population of 1×10⁶. All samples will be filled using an Eppendorf automatic pipette. Two samples will not be sterilized and will be used as positive controls. The samples will be subjected to a variety of sterilization times and temperatures such as those listed in the tables above. Samples will then be analyzed for the presence of active bacteria, or spores by methods such as described above. Conditions suitable for preparation of LDPE containers filled with sterile cyanoacrylates will be identified.

### Prophetic Example III

At least five samples of polymer modified 2-octyl (2-OCA) and n-butyl (n-BCA) cyanoacrylates stabilized with 100 ppm of SO₂ and 1000 ppm of BHA will be filled and sealed in plastic phenolic resin containers with cotton thread biological indicators (BI's) added with a spore population of 1×10⁶. All samples will be filled using an Eppendorf automatic pipette. Two samples will not be sterilized and will be used as positive controls. The samples will be subjected to a variety of sterilization times and temperatures such as those listed in the tables above. Samples will then be analyzed for the presence of active bacteria, or spores by methods such as described above. Conditions suitable for preparation of LDPE containers filled with sterile cyanoacrylates will be identified.

1. A method of sterilization of 2-cyanoacrylate adhesive compositions comprising:
heating said composition in a sealed container comprised of plastic at a temperature of from about 70° C. to about 140° C. for a period of time sufficient to sterilize the composition.

2. A method according to claim 1 wherein said sterilization temperature is from about 70° C. to about 110° C.

3. A method according to claim 1 wherein said sterilization temperature is from about 70° C. to about 100° C.

4. A method according to claim 1 wherein the plastic is selected from the group consisting of HDPE, surfacefluorinated HDPE, LDPE, surface-fluorinated LDPE, polypropylene, surface-fluorinated polypropylene, and phenolic resin.

5. A method according to claim 1 wherein the plastic is surface-fluorinated HDPE.

6. A method as in claim 1 wherein said cyanoacrylate composition comprises one or more 2-cyanoacrylate ester monomers wherein said cyanoacrylate ester monomer is an alkyl, cycloalkyl, fluoroalkyl, fluorocycloalkyl or fluoroalkoxy 2-cyanoacrylate monomer.

7. A method according to claim 3 wherein said cyanoacrylate composition further comprises at least one thickener selected from the group consisting of poly alkyl-2-cyanoacrylates, poly cycloalkyl-2-cyanoacrylates, poly fluoroalkyl-2-cyanoacrylates, poly fluorocycloalkyl-2-cyanoacrylates, poly alkoxyalkyl-2-cyanoacrylates, poly alkoxycycloalkyl-2-cyanoacrylates, poly fluoroalkoxyalkyl-2-cyanoacrylates, poly alkoxycyclofluoroalkyl-2-cyanoacrylates, poly lactic acid and poly glycolic acid.

8. A method according to claim 7 wherein the at least one thickener is a poly alkyl-2-cyanoacrylate.

9. A method according to claim 8 wherein the alkyl group of the poly alkyl-2-cyanoacrylate is selected from the group consisting of straight chain or branched chain C₄ to C₈ hydrocarbons.

10. A method according to claim 8 wherein said thickening polymer is poly octyl-2-cyanoacrylate.

11. A method according to claim 9 wherein the 2-cyanoacrylate composition further comprises a stabilizer.

12. A method according to claim 11 wherein said stabilizer is one or more of an anionic polymerization inhibitor or a free radical polymerization inhibitor.

13. A method according to claim 7 wherein said cyanoacrylate composition further comprises a plasticizer selected from the group consisting tributyl acetyl citrate, dimethyl sebacate, diethyl sebacate, triethyl phosphate, tri-(2-ethylhexyl) phosphate, tricresyl phosphate, glycerol triacetate, glycerol tributyrate, dioctyl adipate, isopropyl myristate, butyl stearate trioctyl trimellitate and dioctyl glutarate.

14. A method according to claim 13 wherein said plasticizer is up to 50% by weight of the cyanoacrylate composition.

15. An article of manufacture comprising a sterile 2-cyanoacrylate ester composition for use in medicine, or surgery, said composition disposed in a sealed container comprised of plastic, said composition sterilized by heating to a temperature of from about 70° C. to about 140° C. for a period of time sufficient to sterilize said composition.

16. A composition according to claim 15 wherein the sterilization temperature is from about 70° C. to about 110° C.

17. A composition according to claim 15 wherein the sterilization temperature is from about 70° C. to about 100° C.

18. The article of manufacture according to claim 15 wherein the plastic is selected from the group consisting of HDPE, surface-fluorinated HDPE, LDPE, surface-fluroinated LDPE, polypropylene, surface-fluorinated polypropylene and phenolic resin.

19. The article of manufacture according to claim 18 wherein the plastic is surface-fluorinated HDPE.

20. A composition according to claim 15 wherein said cyanoacrylate composition comprises one or more 2-cyanoacrylate ester monomers wherein said cyanoacrylate ester monomer is an alkyl, cycloalkyl, fluoroalkyl, fluorocycloalkyl or fluoroalkoxy 2-cyanoacrylate monomer.

21. A composition according to claim 15 wherein the cyanoacrylate composition further comprises at least one thickener selected from the group consisting of poly alkyl-2-cyanoacrylates, poly cycloalkyl-2-cyanoacrylates, poly fluoroalkyl-2-cyanoacrylates, poly fluorocycloalkyl-2-cyanocrylates, poly alkoxyalkyl-2-cyanoacrylates, poly alkoxycycloalkyl-2-cyanoacrylates, poly fluoroalkoxyalkyl-2-cyanoacrylates, poly alkoxycyclofluoroalkyl-2-cyanoacrylates, poly lactic acid and poly glycolic acid.

22. A composition according to claim 21 wherein the thickener is a poly alkyl-2-cyanoacrylate.

23. A composition according to claim 22 wherein the alkyl group of the poly alkyl-2-cyanoacrylate is selected from the group consisting of straight chain or branched chain C₄ to C₈ hydrocarbons.

24. A composition according to claim 23 wherein the thickener is poly octyl-2-cyanoacrylate.

25. A composition according to claim 15 wherein the 2-cyanoacrylate composition further comprises a stabilizer.

26. A composition according to claim 25 wherein said stabilizer on one or more of an anionic polymerization inhibitor or a free radical polymerization inhibitor.

27. A composition according to claim 15 wherein said cyanoacrylate composition further comprises a plasticizer selected from the group consisting of tributyl acetyl citrate, dimethyl sebacate, diethyl sebacate, triethyl phosphate, tri-(2-ethylhexyl) phosphate, tricresyl phosphate, glycerol triacetate, glycerol tributyrate, dioctyl adipate, isopropyl myristate, butyl stearate trioctyl trimellitate and dioctyl glutarate.

28. A composition according to claim 27 wherein said plasticizer is up to 50% by weight of the cyanoacrylate composition.

29. An article of manufacture comprising a sterile 2-cyanoacrylate ester composition for use in medicine, or surgery;
said composition disposed in a sealed container comprised of surface-fluorinated HDPE;
said composition sterilized by heating to a temperature of from about 70° C. to about 140° C. for a period of time sufficient to sterilize said composition;
said composition further comprising a thickener comprised of poly alkyl-2-cyanoacrylate;
said composition further comprising a stabilizer comprised of an anionic polymerization inhibitor and a free radical polymerization inhibitor;
said composition further comprising a plastizer wherein the plasticizer is up to 50% by weight of the cyanoacrylate composition.
